(19) United States
(12) Patent Application Publication
(10) Pub. No.: US 2018/0092774 A1
(43) Pub. Date: Apr. 5, 2018

(54) System, Apparatus and Method for Predicting Anterior Chamber Intraocular Pressure

(71) Applicant: Abbott Medical Optics Inc., Santa Ana, CA (US)

(72) Inventors: Deep Mehta, Irvine, CA (US); Hou Man Chong, La Palma, CA (US); Dung T. Ma, Anaheim, CA (US)

(21) Appl. No.: 15/282,752

(22) Filed: Sep. 30, 2016

(51) Int. Cl. A61F 9/007; A61B 3/16

(52) U.S. Cl. CPC A61F 9/00781; A61B 3/16; A61F 9/00745

(57) ABSTRACT

A system and method for providing the intraocular pressure (IOP) in the anterior chamber of an eye during ocular surgery is provided. Determination of IOP may be performed using an algorithm that is a function of the static (inflow) pressure and/or dynamic (outflow) pressure. Static (inflow) pressure may be calculated as a function of one or more parameters, including bottle height, wound leakage, sleeve size, length of irrigation tubing and/or inner diameter of the irrigation tubing. Dynamic (outflow) pressure may be calculated as a function of one or more parameters, including aspiration rate, vacuum rate, tip size, compliance of tubing, length of tubing, and/or inner diameter of tubing. Additional parameters may also be considered in the algorithm such as patient eye level.

### BACKGROUND

### Field of Invention

The present disclosure relates generally to medical apparatuses and methods that provide pressurized infusion of liquids for ophthalmic surgery, and more particularly, to medical apparatuses and methods that require determinable, stable or controlled intraocular pressure (IOP) within the anterior chamber of the eye.

### Description of Related Art

During ophthalmic surgery, an ophthalmic surgical apparatus is used to perform surgical procedures in a patient's eye. An ophthalmic surgical apparatus typically includes a handheld medical implement or tool, such as a handpiece with a tip, and operating controls for regulating settings or functions of the apparatus and tool. Operation of the tool requires control of various operating settings or functions based on the type of tool used. Such apparatuses typically include a control module, power supply, an irrigation source, one or more aspiration pumps, as well as associated electronic hardware for operating a multifunction handheld surgical tool. The handpiece may include a needle or tip which is ultrasonically driven once placed with the incision to, for example, emulsify the eye lens. In various surgical procedures, these components work together in order to, for example, emulsify eye tissue, irrigate the eye with a saline solution, and aspirate the emulsified lens from the eye.

An exemplary type of ophthalmic surgery is phacoemulsification. Phacoemulsification includes making a corneal and/or scleral incision and the insertion of a phacoemulsification handpiece that includes a needle or tip that is ultrasonically driven to emulsify, or liquefy, the lens. A phacoemulsification system typically includes a handpiece coupled to an irrigation source and an aspiration pump. The handpiece includes a distal tip that emits ultrasonic energy to emulsify a crystalline lens within the patient's eye. The handpiece includes an irrigation port proximal to the distal tip and coupled to the irrigation source via an irrigation input line. The handpiece further includes an aspiration port at the distal tip that is coupled to the aspiration pump via an aspiration output line. Concomitantly with the emulsification, fluid from the irrigation source (which may be a bottle or bag of saline solution that is elevated above the patient's eye, to establish positive pressure by gravity, and/or with external pressure source) is irrigated into the eye via the irrigation line and the irrigation port. This fluid is directed to the crystalline lens in the patient's eye in order to maintain the anterior chamber and capsular bag and replenish the fluid aspirated away with the emulsified crystalline lens material. The irrigation fluid in the patient's eye and the crystalline lens material is aspirated or removed from the eye by the aspiration pump and line via the aspiration port. In some instances, the aspiration pump may be in the form of, for example, a peristaltic or positive displacement pump. Other forms of aspiration pumps are well known in the art, such as vacuum pumps. Additionally, some procedures may include irrigating the eye and aspirating the irrigation fluid without concomitant destruction, alteration or removal of the lens.

Intraocular pressure (IOP) is the fluid pressure inside the anterior chamber of the eye. In a normal eye, intraocular pressure may vary depending on the time of day, activities of the patient, fluid intake, medications, etc. Intraocular pressure may be measured as static (a specific value) or dynamic (a range of values). As can be appreciated, the static IOP and dynamic IOP of a patient's eye can fluctuate greatly during an ophthalmic surgery procedure. It is well known the IOP in an anterior chamber of the eye is required to be controlled and maintained during such surgical procedures in order to avoid damage to the patient's eye. For the correct function of the eye and its structure (e.g. shape) and to preserve sharp and undamaged vision, it is very important to keep the IOP in normal, physiological values.

Different medically recognized techniques have been utilized for ophthalmic surgery, such as phacoemulsification, in order to maintain and control the IOP of a patient's eye. In various examples, phacoemulsification may involve combining irrigation, aspiration and emulsification within a single handpiece. The handpiece that is typically controlled electrically in order to, for example, control the flow of fluid through the handpiece and tip. As may be appreciated, during a surgical procedure, the flow of fluid to and from a patient's eye (through a fluid infusion/irrigation system or aspiration/extraction system, for example), the fluid pressure flowing through the handpiece, and the power control over the handpiece, are all critical to the procedure performed. Precise control over aspiration and irrigation to the ocular region is desired in order maintain a desired or optimal IOP within the anterior chamber of the eye that is similar to the normal pressure within the patient's eye. Similarly, it may be necessary to maintain a stable volume of liquid in the anterior chamber of the eye, which may be accomplished by irrigating fluid into the eye at the same rate as aspirating fluid and lens material from the eye. Accordingly, the ability to predict or determine the static IOP and dynamic IOP of a patient's eye during a surgical operation would be beneficial to a surgeon or operator of such a surgical apparatus.

In prior ophthalmic surgical devices, the control and settings of the system may be electronically controlled or modified by use of a computer system, control module or a user/surgeon. For instance, the control module may also provide feedback information to a user or surgeon regarding the function and operation of the system, or may also receive input from a user or surgeon in order to adjust surgical settings. A surgeon or user may interface with a display system of the control module during use of the device.

Additionally, a surgeon or user may control or adjust certain aspects of the IOP by adjusting various settings or functions of the system. For instance, the irrigation source may be in the form of a suspended or lifted saline bottle or bag, and the surgeon is typically able to adjust the height of the bottle or bag to create a specific head height pressure of the fluid flowing from the bottle bag. In typical systems, the head height pressure, which is a function of the column height, is the static IOP of the fluid flowing through the patient's eye. Accordingly, the surgeon may be able to indirectly set the static IOP by changing the bottle height to a desired level. However, dynamic IOP is a function of surgical parameters and the surgical environment during surgery. Currently, ophthalmic systems do not provide any means for measuring or predicting dynamic IOP.

Based on the foregoing, it would be advantageous to provide a means for determining both the static IOP and dynamic IOP of a patient's eye throughout a surgical operation. Further, it would be advantageous to provide a means for determining a total IOP for the patient's eye from the static IOP, dynamic IOP, and/or other variables of the surgical operation. Such a design would afford a surgeon the ability to perform desired phacoemulsification, diathermy, or vitrectomy functions with better understanding of the surgical environment and process during the surgical procedure.

### SUMMARY

According to one aspect of the present invention, an ocular surgical apparatus includes a process whereby the static IOP and/or dynamic IOP are determined based on various characteristics of the apparatus, the various characteristics either being provided to the system from the surgeon user or collected/determined by various subsystems of the system, the process including determining the static IOP and/or dynamic IOP based on a function of the various characteristics and displaying the static IOP and/or dynamic IOP on a display or screen of the apparatus.

According to another aspect of the present invention, a method of providing an IOP of a patient's eye for selected surgical settings during ocular surgery involves using a predictive IOP algorithm that provides an expected IOP for the surgical settings, the algorithm taking into account various surgical settings that account for fluid inflow and fluid outflow in order to predict a total IOP.

Other systems, methods, features and advantages of the invention will be or will become apparent to one of skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The organization and manner of the structure and function of the disclosure, together with the further objects and advantages thereof, may be understood by reference to the following description taken in connection with the accompanying drawings, and in which:

FIG. 1 illustrates a diagram of an exemplary phacoemulsification/diathermy/vitrectomy system in accordance with the present disclosures, the system including a control module to control various features of the system;

FIG. 2A illustrates an alternative phacoemulsification/diathermy/vitrectomy system and illustrated connected to various components of the system in order to determine characteristics or features of the components;

FIG. 2B illustrates a portion of the alternative phacoemulsification/diathermy/vitrectomy system and illustrated connected to various components of the system in order to determine characteristics or features of the components;

FIG. 3 illustrates an embodiment of a graphical user interface of the system of FIG. 1 or 2, illustrating means for inserting various variables into the control module to permit the control module to determine IOP calculation(s);

FIG. 4 illustrates an embodiment of a graphical user interface of the system of FIG. 1 or 2, further illustrating means for inserting various variables into the control module to permit the control module to determine IOP calculation(s); and

FIG. 5 illustrates an embodiment of a graphical user interface of the system of FIG. 1 or 2, further illustrating for inserting various variables into the control module to permit the control module to determine IOP calculation(s).

### DETAILED DESCRIPTION

The following description and the drawings illustrate specific embodiments sufficiently to enable those skilled in the art to practice the described system and method. Other embodiments may incorporate structural, logical, process and other changes. Examples merely typify possible variations. Individual components and functions are generally optional unless explicitly required, and the sequence of operations may vary. Portions and features of some embodiments may be included in or substituted for those of others.

A system and method for receiving and/or detecting certain variables of a surgical system and utilizing those variables to predict an intraocular pressure (IOP) and/or determine an IOP in real time during a surgical procedure to either provide a notification to a surgeon or allow a target IOP to be set and maintained may be determined by Static IOP, dynamic IOP, and/or a total IOP combining both static and dynamic IOP of the anterior chamber of a patient's eye, which can be applied to any type of system, are disclosed herein. For example, the IOP may be segmented into static and dynamic during a phacoemulsification procedure, static IOP being primarily impacted by the fluid inflow with small amount of outflow and dynamic IOP being primarily impacted by fluid outflow. In illustrative embodiments, the system and method include means for calculating the static IOP, dynamic IOP, and/or total IOP through information provided by a user (e.g. a surgeon) of the system or information collected by a control module of the system. In illustrative embodiments, the system and method include a graphical user interface or other user interface that permits a user to insert information about various components of the system. In other illustrative embodiments, the system can determine various parameters of the system through internal sub-systems (e.g. sensors) to collect information about various components of the system and display such information on the user interface. The system may use such information to calculate the static IOP and/or dynamic IOP of the system, and the total IOP may be function of the static IOP and/or dynamic IOP measurements. In addition, as will be discussed additional parameters or factors may also be considered in determining a total IOP.

As discussed herein, a stable IOP may be of critical importance in order to maintain a stable anterior chamber pressure during phacoemulsification. A stable IOP may be a function of fluid inflow and outflow such that the volume, and in turn the pressure of anterior chamber, remains stable when a chamber is at or near equilibrium.

Embodiments of a subsystem and method will be discussed herein with a particular emphasis on a medical or hospital environment where a surgeon or health care practitioner performs. For example, an embodiment is a phacoemulsification surgical system that comprises an integrated high-speed control module for a phacoemulsification or vitrectomy handpiece that is configured to be inserted into a patient's eye during the phacoemulsification procedure. The system may further comprises one or more sensor(s) to detect variables about the function and operation of the system, such as the rate of fluid flow before and after the fluid flows through the handpiece, and a processor that can collect such variables and/or receive additional variables as inputs from a user, in order to determine the static IOP and dynamic IOP of the anterior chamber of the patient's eye during surgery. The system may further comprise a processor that may control, adjust or set various characteristics of the system to control a phacoemulsification or a high-speed pneumatic vitrectomy handpiece based on the static IOP and/or dynamic IOP measurements determined.

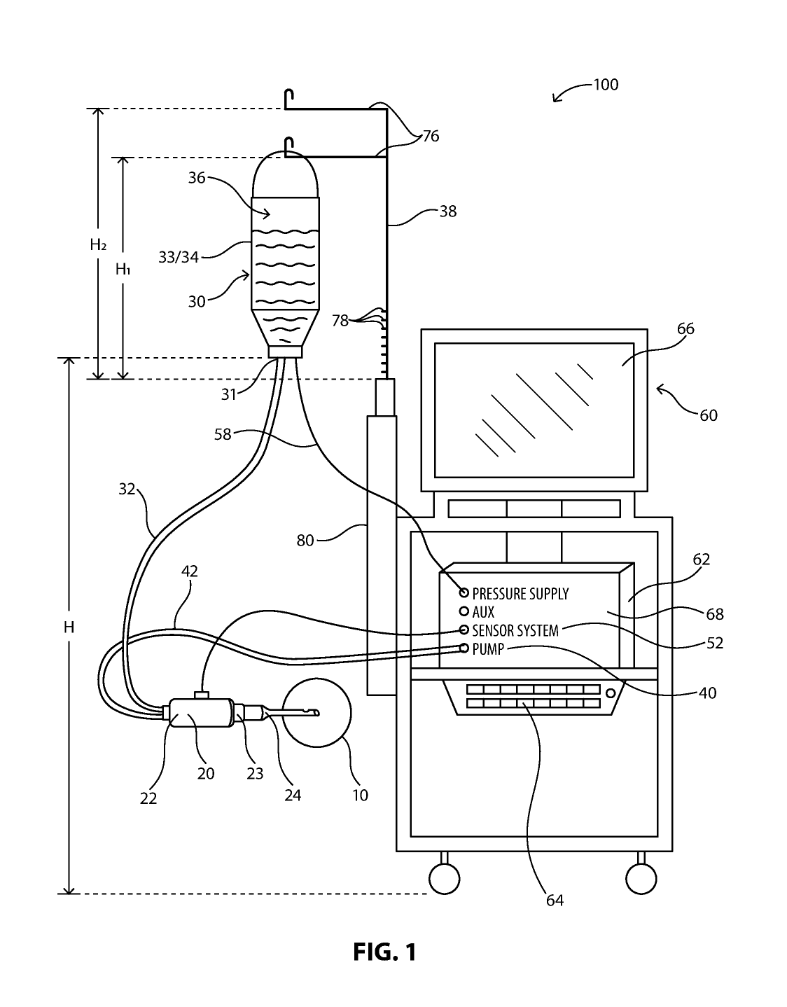

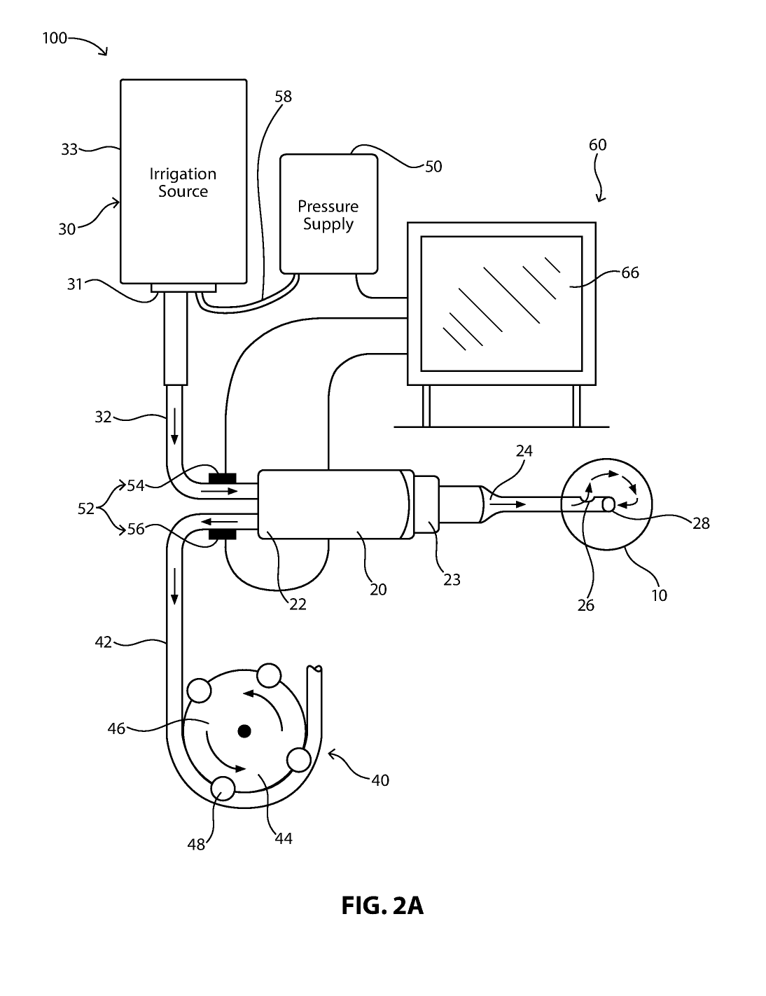

FIGS. 1 and 2 illustrate an exemplary phacoemulsification/diathermy/vitrectomy system 100. As illustrated, the system 100 includes, for example, a handpiece or wand 20, an irrigation source 30, an aspiration source 40, an optional pressure supply 50, and a control module 60. In illustrative embodiments, fluid is controllably directed through the system 100 in order to irrigate a patient's eye, illustrated representatively at 10, during an ocular surgical procedure. Various embodiments of the handpiece 20, irrigation source 30, aspiration source 40, optional pressure supply 50 and control module 60 are well known in the art and are embodied in this disclosure.

As illustrated in FIG. 2A, the irrigation source 30 is configured to supply a predetermined amount of fluid to the handpiece 20 for use during a surgical operation. Such fluid is supplied in order to, for example, stabilize or maintain a certain IOP in the anterior chamber of the eye during surgery, as well as provide means for fluidly transporting any particles (e.g. lens particulates that are created during emulsification) out of the eye. Various aspects (e.g. the flow rate, pressure) of fluid flow into and out of the anterior chamber of the eye will typically affect the operations of the surgical procedure and in particular the IOP measurements of the anterior chamber of the eye during the surgical procedure.

In illustrative embodiments, fluid may flow from the irrigation source 30 to the handpiece 20 via an irrigation line 32. The irrigation source 30 may be any type of irrigation source 30 that can create and control a constant fluid flow. In illustrative embodiments, the irrigation source is elevated to a predetermined height via an extension arm 38. In illustrative embodiments, the irrigation source 30 may be configured to be an elevated drip bag 34 that supplies a steady state of fluid to the irrigation line 32. The pressure supply 50 may be coupled to the irrigation source 30 in order to maintain a constant pressure in the irrigation source 30 as fluid exits the irrigation source 30, as is known in the industry. Other embodiments of a uniform irrigation source are well known in the art.

During the surgical procedure, it is typically necessary to remove or aspirate fluid and other material from the eye. Accordingly, fluid may be aspirated from the patient's eye, illustrated representatively at 10, via the handpiece 20 to flow through an aspiration line 42 to the aspiration source 40. The aspiration source 40 may be any type of aspiration source 40 that aspirates fluid and material from the eye. In illustrative embodiments, the aspiration source 40 may be configured to be a flow-based pump 44 (such as a peristaltic pump) or a vacuum-based pump (such as a Venturi pump) that are well known in the art. The aspiration source 40 may create a vacuum system to pump fluid and/or material out of the eye via the aspiration line 42. Other embodiments of an aspiration source are well known in the art.

The handpiece 20 includes a first end 22 and a second end 23. In various embodiments, the second end 23 may be configured to receive an interchangeable tip 24, which may comprise a sleeve 90 and needle 91, as illustrated as a perspective view in FIG. 2B. The needle 91 can be seen through irrigation port 92 in this view. The needle 91 moves through opening 93 and is employed to break up the cataract. Irrigation port 26 is located on one side of the sleeve 90, while a second port is not shown but may be located on the other side of the sleeve 90. Multiple ports may be provided, including more than two ports, while still within the scope of the present invention.

The irrigation port 26 is fluidly coupled to the irrigation line 32 to receive fluid flow from the irrigation source 30, and the aspiration port 28 is fluidly coupled to the aspiration line 42 to receive fluid and/or material flow from the eye. The handpiece 20 and the tip 24 may further emit ultrasonic energy into the patient's eye, for instance, to emulsify or break apart the crystalline lens within the patient's eye. Such emulsification may be accomplished by any known methods in the industry, such as, for example, a vibrating unit (not shown) that is configured to ultrasonically vibrate and/or cut the lens, as is known in the art. Other forms of emulsification, such as a laser, are well known in the art. Concomitantly with the emulsification, fluid from the irrigation source 30 is irrigated into the eye via the irrigation line 32 and the irrigation port 26. During and after such emulsification, the irrigation fluid and emulsified crystalline lens material are aspirated from the eye by the aspiration source 40 via the aspiration port 28 and the aspiration line 42. Other medical techniques for removing a crystalline lens also typically include irrigating the eye and aspirating lens parts and other liquids. Additionally, other procedures may include irrigating the eye and aspirating the irrigating fluid within concomitant destruction, alternation or removal of the lens.

The aspiration source 40 is configured to aspirate or remove fluid and other materials from the eye in a steady, uniform flow rate. Various means for steady, uniform aspiration are well known in the art. In illustrative embodiments, the aspiration source 40 may be a Venturi pump, a peristaltic pump, or a combined Venturi and peristaltic pump. In illustrative embodiments, and as shown in FIG. 2, a peristaltic pump 44 may be configured to include a rotating pump head 46 having rollers 48. The aspiration line 42 is configured to engage with the rotating pump head 46 as it rotates about an axis. As the pump head 46 rotates the rollers 48 press against the aspiration line 42 causing fluid to flow within the aspiration line 42 in a direction of the movement for the rollers 48. Accordingly, the pump 44 directly controls the volume or rate of fluid flow, and the rate of fluid flow can be easily adjusted by adjusting the rotational speed of the pump head 46. Other means of uniformly controlling fluid flow in an aspiration source 40 are well known in the art. When the aspiration source 40 includes a combined Venturi and peristaltic pump, the aspiration source 40 may be controlled to automatically switch between the two types of pumps or user controlled to switch between the two types of pumps.

In illustrative embodiments, the control module 60 is configured to monitor and control various components of the system 100. For instance, the control module 60 may monitor, control, and provide power to the pressure supply 50, the aspiration source 40, and/or the handpiece 20. The control module 60 may be in a variety of forms as known in the art. In illustrative embodiments, the control module 60 may include a microprocessor computer 62, a keyboard 64, and a display or screen 66, as illustrated in FIGS. 1, 2A, 3, 4 and 5. The microprocessor computer 62 may be operably connected to and control the various other elements of the system, while the keyboard 64 and display 66 permit a user to interact with and control the system components as well. In illustrative embodiments, the control module 60 may also include a pulsed ultrasonic power source (not shown) that can be controlled by the computer 62 in accordance with known methods or algorithms in the art. A system bus 68 may be further provided to enable the various elements to be operable in communication with each other.

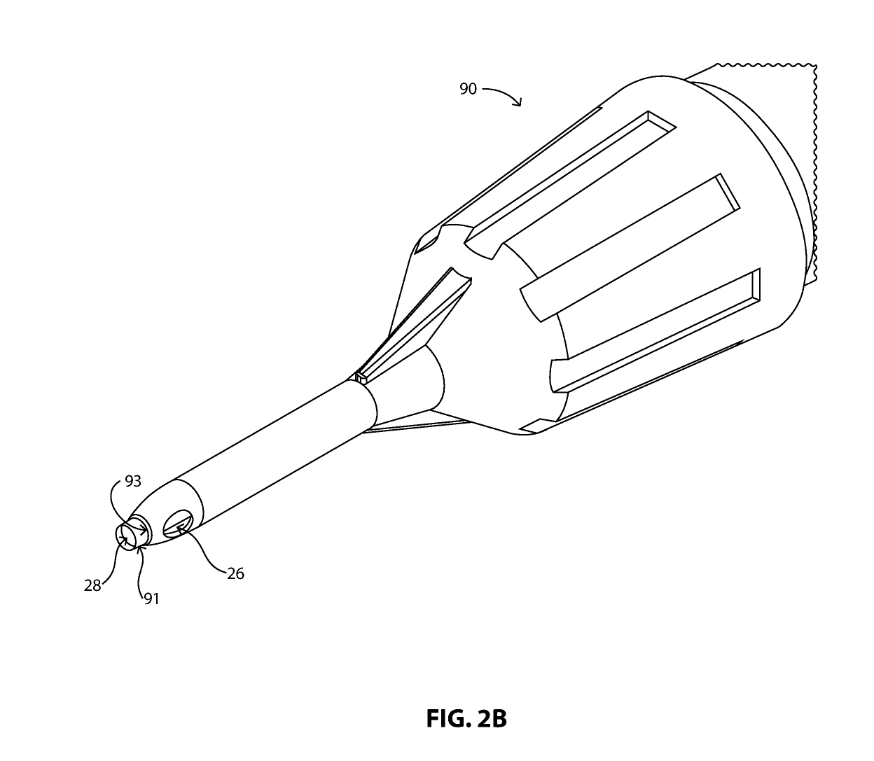

The screen 66 may display various measurements, criteria or settings of the system 100—such as the type of procedure, the phase of the procedure and duration of the phase, various parameters such as vacuum, flow rate, power, and values that may be input by the user, such as bottle height, sleeve size, tube length (irrigation and aspiration), tip size, vacuum rate, etc., as illustrated in FIGS. 3-5. The screen 66 may be in the form of a graphical user interface (GUI) 70 associated with the control module 60 and utilizing a touchscreen interface, for example. The GUI 70 may allow a user to monitor the characteristics of the system 100 or select settings or criteria for various components of the system. For instance, the GUI 70 may permit a user to select or alter the maximum pressure being supplied by the pressure supply 50 to the irrigation source 30. The user may further control the operation of the phase of the procedure, the units of measurement used by the system 100, or the height of the irrigation source 30, as discussed below. The GUI 70 may further allow for the calibration and priming of the pressure in the irrigation source 30.

In illustrative embodiments, the system 100 may include a sensor system 52 configured in a variety of ways or located in various locations. For example, the sensor system 52 may include at least a first sensor or strain gauge 54 located along the irrigation line 32 and a second sensor or strain gauge 56 located along the aspiration line 42, as illustrated in FIG. 2. Other locations for the sensors 54 and 56 are envisioned anywhere in the system 100 and may be configured to determine a variety of variables that may be used to determine IOP measurements in the eye, as discussed below. This information may be relayed from the sensor system 52 to the control module 60 to be used in the determination of IOP measurements. The sensor system 52 may also include sensors to detect other aspects of the components used in the system, e.g. type of pump used, type of sleeve used, gauge of needle tip (size), etc.

In order to determine the IOP of a patient's eye during surgery, the system 100 may be configured to determine and/or receive a variety of variables about the system 100 that may be used in a predictive algorithm to determine the IOP range before the surgery begins or provide the IOP during surgery based on the entered parameters and/or sensed parameters. The algorithm may be performed on the control module 60 and takes into account one or more of the parameters, such as bottle height, tip size, sleeve size, aspiration rate, vacuum rate, length and compliance metrics of various tubing used in the system, and/or pump rate, as will be described below. Other parameters for consideration in the algorithm are envisioned within the scope of this disclosure. Specifically, the following algorithms alone or in combination in addition to other parameters discussed below may be used to determine IOP measurements:
Static IOP=function {bottle height, wound leakage, sleeve size, length of irrigation tubing, and/or inside diameter of irrigation tubing}
Dynamic IOP=function {tip size, aspiration rate, vacuum rate, aspiration tubing length, inside diameter of aspiration tubing, tubing compliance,}
Other parameters to consider: patient eye level
   The variables of these algorithms will now be discussed.

One factor for consideration in the determination of IOP measurement is the bottle height of the irrigation source 30. As illustrated in FIG. 1, the irrigation source 30, specifically the exit port 31 of the irrigation source, is typically elevated to a predetermined height H. This predetermined elevation may be accomplished by any known means. For example, the irrigation source 30 may be connected to one or more fixed supports 76 on the extension arm 38, the fixed supports spaced at varying heights H1 and H2 along the extension arm 38 to permit the irrigation source 30 to hang down via the force of gravity and place the exit port 31 of the irrigation source 30 at predetermined height H. Alternatively, the extension arm 38 may be retractable (or movable) relative to a fixed receiver 80, the extension arm 38 including biased retaining members 78 that can engage with an aperture (not shown) of the fixed receiver 80 to maintain the extension arm 38 in a relative position with respect to the fixed receiver 80. In such an embodiment, the height H of the exit port 31 of the irrigation source 30 (with respect to the ground) may be maintained in the predetermined position based on the specific retaining member 78 engaging with the aperture of the fixed receiver 80, as is known in the art. Other means of height adjustment are known in the art.

The bottle height may be inputted manually into the control module 60 of the system by a user via a graphical user interface 70. Alternatively, the bottle height may be determined by the control module 60 automatically and displayed on the graphical user interface 70. For example, a sensor system (not shown) may be connected to the extension arm 38 or the fixed receiver 80 to determine the height H of the exit port 31.

Another factor for consideration in the determination of IOP measurement is the size of a sleeve 90 around needle 24. The size of a sleeve or dimension of a sleeve can be a factor in the amount of fluid that flows from irrigation source 30 into the eye.

The size of the sleeve 90 may be inputted manually into the control module 60 of the system by a user via a graphical user interface 70. Alternatively, the size of the sleeve 90 may be determined automatically by the control module 60 and displayed on the graphical user interface 70. For example, a sensor system (not shown) may be connected to the handpiece 20 and/or near the sleeve 90. During a calibration or prime and tune cycle of the system 100, the system may be able to determine the size of the sleeve 90 by comparing information received from the sensor, e.g. on the amount of fluid flow out of the sleeve based on the height of the bottle or the flow rate if a pressurized system is used. For example, the sleeve size may be based on the gauge of the needle used or selected independently of the needle selected. According to an embodiment, the sleeve size may be selected from Table 1 below having the corresponding dimension:

TABLE 1

| Sleeve | Incision Size | Type |
|---|---|---|
| OPOS19L | 3/3.25 mm | 19Ga (Blue) |
| OPOS20L | 2.4/2.7 mm | 20Ga (Yellow) |
| OPOS21L | 2.1/2.4 mm | 21Ga (Light Blue) |
| OPOHF20L | 2.75/3.0 mm | 20Ga High Flow (Purple) |
| OPOHF21L | 2.4/2.7 mm | 21Ga High Flow (Orange) |

In an embodiment of the present invention, where a basic level of IOP is to be determined, a static IOP may be a function of fluid inflow only, where fluid inflow is governed by the column height of the bottle (bottle height) and wound leakage. As discussed herein, column height of the fluid may be governed by IV bottle height or other pressurizing source such as, for example, vented gas forced infusion (VGFI) and/or incision wound leakage. By way of example, as the height of the IV bottle changes, the static IOP would change accordingly. A surgeon may therefore set the desired static IOP before and during the surgery by adjusting the bottle height or other pressurizing source. The pressure exerted by the bottle height of the fluid is governed by following function:

Pressure (mmHg)=Column Height (cm)×10/density of the fluid (for example, the fluid may be mercury, the density of which is assumed to be 13.6 g/cm³, or water, which has a density of 1 g/cm³).

Assuming that the amount of wound leakage is governed by the size of incision, the static IOP=function {Bottle Height, Wound Leakage}. Using this function, the system of the present invention may determine the bottle height by measuring the IV pole height or input pressure of the pressurizing source.

In another embodiment of the present invention, additional parameters are considered in determining IOP is described. During phacoemulsification, for example, the IOP of the anterior chamber may vary as lens fragments are emulsified and aspirated from the anterior chamber of the eye. Such variability is a result of the dynamic nature of intraocular pressure during the phacoemulsification procedure. In an embodiment of the present invention, dynamic IOP may be governed by the flow or aspiration rate. During a procedure, flow and vacuum rates may change as fragments are emulsified and aspirated. This may cause changes in volume of the anterior chamber which in turn may cause the IOP to vary. Thus, the Total IOP may take into account Static IOP parameters and Dynamic IOP parameters. The Total IOP may be predicted before the surgery and also may be determined during surgery based on real time measurements. In addition, a target IOP a surgeon would like to maintain in a patient's eye may be set and the system may adjust various system components or parameters based on sensed data used to calculate periodic Total IOPs during the surgery to maintain the target IOP. Thus, based on the example described above, the Total IOP=Static IOP function {Bottle Height, Wound Leakage}+Dynamic IOP function {Flow Rate/Aspiration Rate}. Using this algorithm, the system of the present invention may determine the bottle height by measuring IV pole height or input pressure of the pressurizing source. Wound leakage may be determined by the size of the incision made during the surgery. Further, the system of the present invention may determine fluid outflow by measuring the flow rate at the aspiration line. Moreover, based on the system calculating the Total IOP at various time points before and/or during the procedure a target IOP may be maintained by adjusting one or more parameters, e.g. irrigation flow rate, bottle height, aspiration rate, etc.

In another embodiment of the present invention, a predictive IOP algorithm may be further optimized to provide a more accurate prediction of Total IOP during surgery by taking into account additional parameters, such as the tip and sleeve sizes along with the compliance and length of the associated tubing apparatus. Such an algorithm may use the above stated parameters to provide one or more IOP measurements before and/or during surgery. Thus, the Total IOP=Static IOP {fluid inflow}+Dynamic IOP {fluid outflow}; wherein fluid inflow=function {bottle height or input pressure, sleeve size, wound leakage, length of the irrigation tubing, and inside diameter of irrigation tubing} and fluid outflow=function {flow/aspiration rate, vacuum rate, tip size, compliance, length and inside diameter of aspiration tubing}.

Bottle height may be obtained by measuring the IV pole height and/or input pressure of the pressurizing source and the patient eye level is input prior to the surgery and does not change during the surgery. Generally, the sleeve size is fixed during the surgery and a user may either provide the type and size of the sleeve used or the system may infer this value by measuring the out flow from the sleeve during the prime and tune processes. Flow rate may be variable during a surgery and the system may obtain a flow rate value by measuring the flow of fluid through the aspiration line, while a maximum aspiration rate is generally preset by a user prior to starting a surgery. The vacuum rate may be variable during the surgery and the system may obtain a value for the vacuum rate by measuring the running vacuum during surgery. The length of the irrigation/aspiration (I/A) tubing is generally defined as the distance from the end of the hand piece to the pack or cassette. The system may infer this value from the type of pack used. Compliance of the I/A tubing may be defined for each type of pack such as, for example, a single-use pack or multi-use pack. The system may infer this value from type of pack used. Similarly, the inside diameter of the I/A tubing may be defined for each type of pack, which value may be inferred from type of pack used.

Another factor for consideration in the determination of IOP measurement is tip size. In illustrative embodiments, the tip 24 of the handpiece 20 may be interchangeable with several other interchangeable tips 24 that have different features or characteristics. These tips 24 may have predetermined or uniform shapes and port sizes/locations based on the specific tip selected, so that a certain tip size is an industry standard and is known to have industry standard dimensions and features. Each of the different tip sizes may include or provide benefit in the way of different features that assist with performing the surgical operation. Such tips 24 are generally known to be of uniform sizes or types in the industry, such that certain tips 24 may be considered advantageous for certain surgical maneuvers or operations. Tips of uniform size or type may be identified by specific name or product number to be an industry standard design. Surgeons or other users of such tips may have industry knowledge of the types of tips available and their varying characteristics, and may rely on the uniformity of tip types from operation to operation.

The tip size may be inputted manually into the control module 60 of the system 100 by a user via the graphical user interface 70. Alternatively, the tip size may be determined by the control module 60 automatically and displayed on the graphical user interface 70. In this regard, applicant refers to U.S. Patent Application No. 62/293,283, incorporated by reference herein.

Other factors for consideration in the determination of IOP measurement are the characteristics of the irrigation and aspiration lines 32, 42 (e.g. tubing). As illustrated in FIGS. 1 and 2, the irrigation line 32 connects the irrigation source 30 to the handpiece 20 and delivers fluid to the handpiece 20, and the aspiration line 42 connects the handpiece 20 to the aspiration source 40 and removes fluid from the eye via the handpiece 20. These lines 32, 42 typically comprise flexible tubing that permits a wide variety of relative movement of the handpiece 20 with respect to the irrigation source 30 and aspiration source 40. The flexible tubing selected for the system may include a variety of lengths and diameters. The length of tubing between the irrigation source 30 and the handpiece 20, and the handpiece 20 and the aspiration source 40, affects the fluid flow and fluid pressure when the fluid enters/leaves the patient's eye. Similarly, the inside diameter of the tubing affects the fluid flow and fluid pressure when the fluid enters/leaves the eye. Similar to the dimensions of the irrigation line 32 and aspiration line 42, the composition of the lines may also affect the flow or pressure of fluid into and out of the patient's eye. Tubing is typically required to meet certain industry standards of compliance, and further be identified based on the compliance requirements of the tubing. The composition (e.g. type of material used to form the tubing) may have certain characteristics (such as compression strength, pliability, etc.) that affect the fluid flow and fluid pressure of fluid flowing into or leaving the patient's eye.

The characteristics of the irrigation and aspiration tubing may be inputted manually into the control module 60 of the system 100 by a user via the graphical user interface 70. Alternatively, a user may be able to select specific tubing based on predetermined requirements identified by the system 100 once a desired IOP is determined. A sensor system (not shown) may exist to determine the compliance, length and diameter of the tubing, alternatively.

Other factors for consideration in the determination of IOP measurement are the characteristics of the aspiration source 40, including the aspiration rate (e.g. rate fluid is aspirated from the eye), vacuum rate (e.g. rate of the vacuum in the aspiration source). The characteristics of the aspiration source 40 may be inputted manually into the control module 60 of the system 100 by a user via the graphical user interface 70, may be preprogrammed in the system, or may be determined by a sensor system (not shown) located along the aspiration line 42 or in the aspiration source 40.

In any of the embodiments described herein, any number of the parameters may be used or values entered by a user or considered by the algorithm when calculating a Total IOP. The more parameters used the more accurate the Total IOP is likely to be.

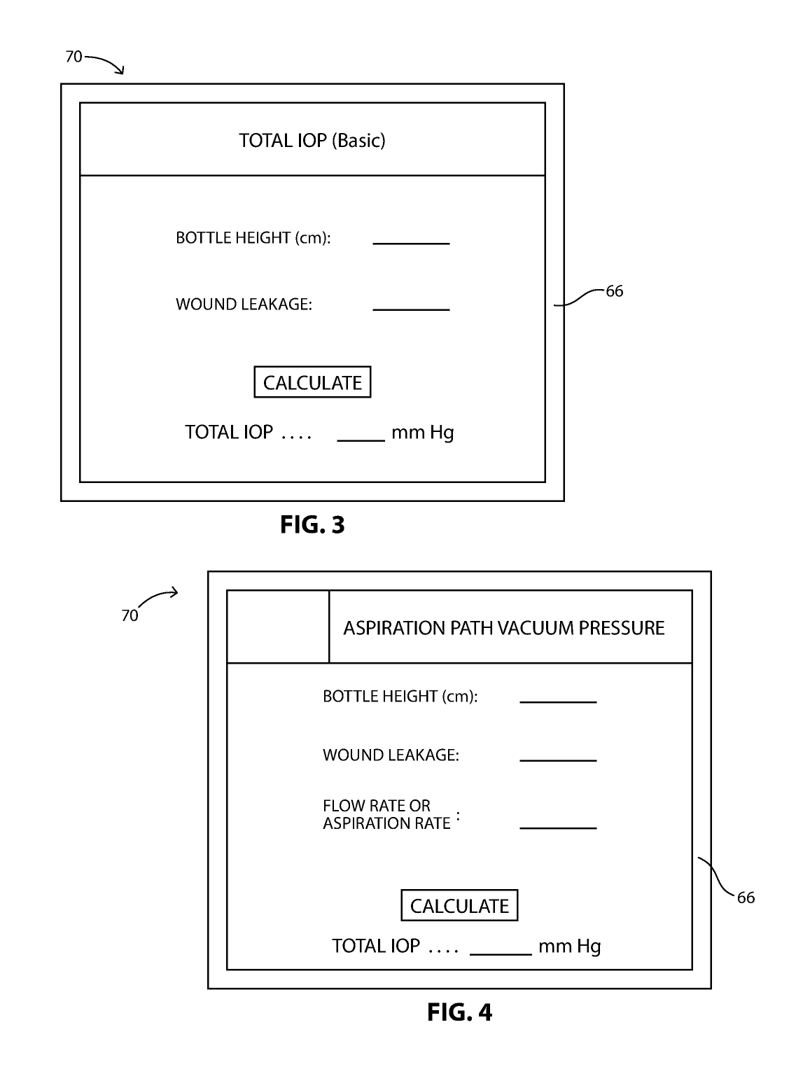

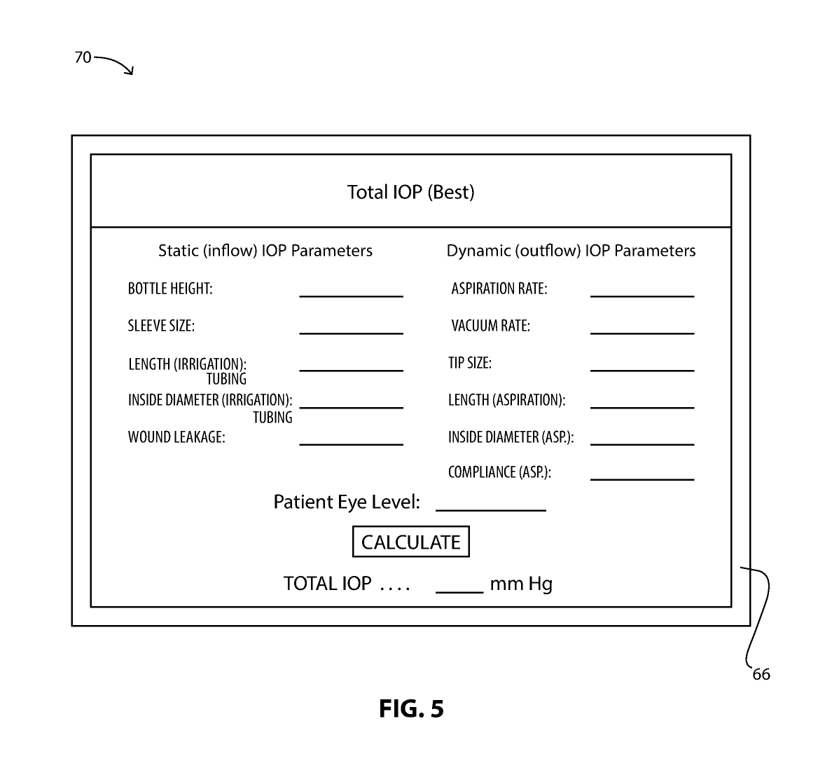

FIGS. 3-5 illustrate exemplary embodiments of a graphical user interface 70 that permits collection and/or display of variables that can affect the static IOP and dynamic IOP when calculating a Total IOP measurement. A variety of methods of collecting and/or displaying information may be encompassed in this disclosure. For example, FIG. 3 illustrates a single-screen IOP calculation on GUI 70, permitting a user to insert two parameters related to the algorithms to calculate a basic Total IOP measurement, as discussed above, and presenting the resulting Total IOP measurement determined from the algorithm calculation. FIG. 4 illustrates a single-screen IOP calculation on GUI 70, permitting a user to insert values for the parameters for both Static IOP and Dynamic IOP to calculate a Total IOP measurement as discussed above, and presenting the resulting Total IOP measurement determined from the algorithm calculation. FIG. 5 illustrates a single-screen IOP calculation on GUI 70, permitting the user to input values for one or more parameters for the Static IOP and/or Dynamic IOP, in addition to the option of entering the patient eye level, to calculate a Total IOP measurement discussed above, and presenting the resulting Total IOP measurement determined from the algorithm calculation. It is also envisioned that each time an IOP calculation is made during surgery it is displayed to a user. In an embodiment, should a target IOP or target range of IOPs be set by a user a visual and/or audible signal can be made to alert the user when the measured IOP falls outside a preset range of the selected or preselected target IOP or target range of IOPs. In addition, to the signal, the system may automatically adjust system parameters to bring the patient's IOP back to the target IOP or within the selected target range of IOP. Those of skill in the art will recognize that any step of a method described in connection with an embodiment may be interchanged with another step without departing from the scope of the invention. Those of skill in the art would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The various illustrative logical blocks, modules, and circuits described in connection with the embodiments disclosed herein may be implemented or performed using a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Any options available for a particular medical device system may be employed with the present invention. For example, with a phacoemulsification system the available settings may include, but are not limited to, irrigation, aspiration, vacuum level, flow rate, pump type (flow based and/or vacuum based), pump speed, ultrasonic power (type and duration, e.g. burst, pulse, duty cycle, etc.), irrigation source height adjustment, linear control of settings, proportional control of settings, panel control of settings, and type (or “shape”) of response.

In illustrative embodiments, the interface provides feedback to the user should the predetermined or automatic settings, variables, or criteria need adjustment to ensure all the desired settings of the system. The interface can then permit the user to change or modify those settings accordingly.

Other mechanisms for setting and/or programming a particular setting may be employed with the present invention, including, but not limited to, clicking on an icon on a display screen using a mouse or touch screen, depressing a button/switch on a foot pedal, voice activated commands and/or combinations thereof.

The term “phacoemulsification” refers to a method of lens and cataract extraction from an eye. The procedure includes an ultrasonically vibrated needle which is inserted through a very small incision in the cornea in order to provide energy for emulsifying or breaking up of the lens and cataract which then can be aspirated and removed through the incision.

The term “vitrectomy surgery” refers to a method employed during cataract surgery when the posterior capsular bag has been broken and in the treatment of retinal detachments resulting from tears or holes in the retina. In cataract surgery, the same incision used for the phacoemulsification handpiece is used for inserting the vitrector to remove the vitreous gel. Vitrectomy surgery typically involves removal of vitreous gel and may utilize three small incisions in the pars plana of the patient's eye. These incisions allow the surgeon to pass three separate instruments into the patient's eye to affect the ocular procedure. The surgical instruments typically include a vitreous cutting device, an illumination source, and an infusion/aspiration port(s), but these devices may be combined into one single tool as well.

The term “screen,” “display,” or “display screen” as used herein shall mean a graphical user interface (GUI), a screen, monitor, touch screen, or any other device known in the art for displaying a visual picture or representation to a user.

### Items of the Present Disclosure

1. A method of calculating an intraocular pressure for a patient's eye, comprising:

applying a predictive algorithm that is predictive of the intraocular pressure by at least one computing processor of a surgical system using code accessed from at least one computing memory associated with the at least one computing processor, the predictive algorithm comprising:
estimating a static pressure as a function of a fluid inflow parameter of the surgical system;
estimating a dynamic pressure as a function of a fluid outflow parameter of the surgical system, and
calculating the intraocular pressure based on the estimated static pressure and the estimated dynamic pressure.

2. A method of calculating an intraocular pressure for a patient's eye, comprising:

applying a predictive algorithm that is predictive of the intraocular pressure by at least one computing processor of a surgical system using code accessed from at least one computing memory associated with the at least one computing processor, the predictive algorithm comprising:
estimating a static pressure as a function of at least two fluid inflow parameters of the surgical system; and
calculating the intraocular pressure based on the estimated static pressure.

3. The method of 2, further comprising estimating a dynamic pressure as a function of one or more fluid outflow parameters of the surgical system, and calculating the intraocular pressure based on the estimated static pressure and the estimated dynamic pressure.

4. The method of 1 or 3, wherein the fluid outflow parameter is one or more selected from the group consisting of aspiration rate, vacuum rate, tip size, a compliance of tubing, a length of tubing, and an inside diameter of tubing.

5. The method of 1, 3 or 4, further comprising estimating a patient eye level for combination with the estimated static pressure and the estimated dynamic pressure for calculating the intraocular pressure.

6. The method of any of 1 or 3 to 5, further comprising automatically determining one or more of the fluid inflow parameters using a sensor system.

7. The method of any of 1 or 3 to 6, wherein at least one of the fluid outflow parameter is inferred.

8. The method of 7, wherein the inferred fluid outflow parameter is inferred from a type of surgical pack used in the surgical system.

9. The method of any of 1 or 3 to 8, further comprising automatically determining one or more of the fluid outflow parameters using a sensor system.

10. The method of any of 1 or 3 to 9, wherein the dynamic pressure is a function of a plurality of surgical settings associated with the surgical system via the at least one processor.

11. The method of any of 1 or 3 to 9, wherein the estimated dynamic pressure is further estimated as a function of the stage of a phacoemulsification of the patient's eye performed by the surgical system.

12. The method of any preceding item, further comprising receiving at a control module associated with the at least one processor and communicative with the predictive algorithm at least one user input comprising one or more of the fluid inflow parameter and fluid outflow parameter.

13. The method of 12, wherein the control module comprises a graphical user interface for the receiving.

14. The method of 13, further comprising displaying, using the graphical user interface, the calculated intraocular pressure.

15. The method of 14, wherein the calculated intraocular pressure comprises a particular value.

16. The method of 13, 14 or 15, wherein the graphical user interface comprises a single screen for said receiving and said displaying.

17. The method of any preceding item, wherein the surgical system comprises a phacoemulsification surgical system.

18. The method of any preceding item, wherein the intraocular pressure varies for each sub-mode setting of the surgical system.

19. The method of any preceding item, wherein the fluid inflow parameter is one or more selected from the group consisting of irrigation inflow pressure (bottle height), wound leakage, sleeve size, tubing length, and inside diameter of tubing.

20. The method of any preceding item, comprising controlling the surgical system so as to achieve a target intraocular pressure based on the calculated intraocular pressure.

21. The method of 20, comprising adjusting irrigation flow rate, irrigation inflow pressure, vacuum rate, and/or aspiration rate of the surgical system based on the calculated intraocular pressure in order to achieve the target intraocular pressure.

22. A system for calculating an intraocular pressure for a patient's eye while the patient's eye is subjected to a surgical system, wherein:

the system comprises the surgical system, which comprises at least one computing processor configured to access code from at least one computing memory associated with the at least one computing processor, the processor thereby configured to:

estimate a static pressure as a function of a fluid inflow parameter of the surgical system; and

estimate a dynamic pressure as a function of a fluid outflow parameter of the surgical system,

calculate the intraocular pressure based on the estimated static pressure and the estimated dynamic pressure.

23. The system of 22, wherein the fluid inflow parameter is one or more selected from the group consisting of bottle height, wound leakage, sleeve size, tubing length, and inside diameter of tubing.

24. The system of 22 or 23, wherein the fluid outflow parameter is one or more selected from the group consisting of aspiration rate, vacuum rate, tip size, a compliance of tubing, a length of tubing, and an inside diameter of tubing.

25. The system of 22, 23 or 24, further comprising a sensor system for determining one or more of the fluid outflow parameters.

26. The system of 22, 23, 24 or 25 further comprising a sensor system for determining one or more of the fluid inflow parameters.

27. The system of any of 22 to 26, wherein the surgical system comprises a phacoemulsification surgical system.

28. A system for calculating an intraocular pressure for a patient's eye while the patient's eye is subjected to a surgical system wherein:

the system comprises the surgical system, which comprises at least one computing processor configured to access code from at least one computing memory associated with the at least one computing processor, the processor thereby configured to:
estimate a static pressure as a function of at least two fluid inflow parameters of the surgical system; and
calculate the intraocular pressure based on the estimated static pressure.

29. The system of 28, wherein the fluid inflow parameter is selected from the group consisting of bottle height, wound leakage, sleeve size, tubing length, and inside diameter of tubing.

30. The system of 28 or 29, the processor further configured to estimate a dynamic pressure as a function of one or more fluid outflow parameters of the surgical system, and calculate the intraocular pressure based on the estimated static pressure and the estimated dynamic pressure.

31. The system of 30, wherein the fluid outflow parameter is one or more selected from the group consisting of aspiration rate, vacuum rate, tip size, a compliance of tubing, a length of tubing, and an inside diameter of tubing.

32. The system of 28, 29, 30 or 31, further comprising a sensor system for determining at least one of the fluid inflow parameters.

33. The system of any of 28 to 32, further comprising a sensor system for determining at least one of the fluid outflow parameters.

34. The system of any of 22 to 33, wherein the surgical system comprises a handpiece combining irrigation, aspiration and emulsification capabilities.

35. The system of any of 22 to 34, wherein the processor is configured to control the surgical system so as to achieve a target intraocular pressure based on the calculated intraocular pressure.

36. The system of claim 35, wherein the processor is configured to control the surgical system so as to achieve the target intraocular pressure based on the calculated intraocular pressure by adjusting any one or more of irrigation flow rate, irrigation inflow pressure, vacuum rate and aspiration rate of the surgical system.

37. The system of any of 22 to 36, wherein the processor is configured to repeatedly calculate the intraocular pressure.

38. The system of any of 22 to 37, wherein the processor is configured to repeatedly calculate the intraocular pressure based at least on sensed aspiration rate of the surgical system.

The previous description is provided to enable any person skilled in the art to make or use the disclosed embodiments. Various modifications to these embodiments will be readily apparent to those skilled in the art, and the generic principles defined herein may be applied to other embodiments without departing from the spirit or scope of the invention. Thus, the present disclosure is not intended to be limited to the embodiments shown herein but is to be accorded the widest scope consistent with the principles and novel features disclosed herein.

What is claimed is:

1. A method of providing an intraocular pressure for a patient's eye while the patient's eye is subjected to a surgical system, comprising:
applying a predictive algorithm that is predictive of the intraocular pressure by at least one computing processor of the surgical system using code accessed from at least one computing memory associated with the at least one computing processor, the predictive algorithm comprising:
estimating a static pressure as a function of a fluid inflow parameter of the surgical system;
estimating a dynamic pressure as a function of a fluid outflow parameter of the surgical system; and
calculating the intraocular pressure based on the estimated static pressure and the estimated dynamic pressure.

2. The method of claim 1, wherein the fluid inflow parameter is one or more selected from the group consisting of bottle height, wound leakage, sleeve size, tubing length, and inside diameter of tubing.

3. The method of claim 1, wherein the fluid outflow parameter is one or more selected from the group consisting of aspiration rate, vacuum rate, tip size, a compliance of tubing, a length of tubing, and an inside diameter of tubing.

4. The method of claim 1, further comprising automatically determining one or more of the fluid outflow parameters using a sensor system.

5. The method of claim 1, further comprising automatically determining one or more of the fluid inflow parameters using a sensor system.

6. The method of claim 1, further comprising receiving at a control module associated with the at least one processor and communicative with the predictive algorithm at least one user input comprising one or more of the fluid inflow parameter and fluid outflow parameter.

7. The method of claim 6, wherein the control module comprises a graphical user interface for the receiving.

8. The method of claim 7, further comprising displaying, using the graphical user interface, the calculated intraocular pressure.

9. The method of claim 8, wherein the calculated intraocular pressure comprises a particular value.

10. The method of claim 8, wherein the graphical user interface comprises a single screen for said receiving and said displaying.

11. The method of claim 1, further comprising estimating a patient eye level for combination with the estimated static pressure and the estimated dynamic pressure for calculating the intraocular pressure.

12. The method of claim 1, wherein the dynamic pressure is a function of a plurality of surgical settings associated with the surgical system via the at least one processor.

13. The method of claim 1, wherein the intraocular pressure varies for each sub-mode setting of the surgical system.

14. The method of claim 1, wherein the estimated dynamic pressure is further estimated as a function of the stage of a phacoemulsification of the patient's eye performed by the surgical system.

15. The method of claim 1, wherein at least one of the fluid outflow parameter is inferred.

16. The method of claim 15, wherein the inferred fluid outflow parameter is inferred from a type of surgical pack used in the surgical system.

17. The method of claim 1, wherein the surgical system comprises a phacoemulsification surgical system.

18. A method of providing an intraocular pressure for a patient's eye while the patient's eye is subjected to a surgical system, comprising:
applying a predictive algorithm that is predictive of the intraocular pressure by at least one computing processor of the surgical system using code accessed from at least one computing memory associated with the at least one computing processor, the predictive algorithm comprising:
estimating a static pressure as a function of at least two fluid inflow parameters of the surgical system; and
calculating the intraocular pressure based on the estimated static pressure.

19. The method of claim 18, wherein the fluid inflow parameter is selected from the group consisting of bottle height, wound leakage, sleeve size, tubing length, and inside diameter of tubing.

20. The method of claim 18, further comprising estimating a dynamic pressure as a function of one or more fluid outflow parameters of the surgical system, and calculating the intraocular pressure based on the estimated static pressure and the estimated dynamic pressure.

21. The method of claim 20, wherein the fluid outflow parameter is one or more selected from the group consisting of aspiration rate, vacuum rate, tip size, a compliance of tubing, a length of tubing, and an inside diameter of tubing.

22. The method of claim 20, further comprising estimating a patient eye level for combination with the estimated static pressure and the estimated dynamic pressure for calculating the intraocular pressure.

23. The method of claim 18, further comprising automatically determining at least one of the fluid inflow parameters using a sensor system.

24. The method of claim 20, further comprising automatically determining at least one of the fluid outflow parameters using a sensor system.

25. The method of claim 20, wherein at least one of the fluid outflow parameters is inferred.

26. The method of claim 25, wherein the inferred fluid outflow parameter is inferred from a type of surgical pack used in the surgical system.

27. A system for calculating an intraocular pressure for a patient's eye while the patient's eye is subjected to a surgical system wherein:
the system comprises the surgical system, which comprises at least one computing processor configured to access code from at least one computing memory associated with the at least one computing processor, the processor thereby configured to:
estimate a static pressure as a function of at least two fluid inflow parameters of the surgical system; and
calculate the intraocular pressure based on the estimated static pressure.

28. The system of claim 27, wherein the fluid inflow parameter is selected from the group consisting of bottle height, wound leakage, sleeve size, tubing length, and inside diameter of tubing.

29. The system of claim 28, the processor further configured to estimate a dynamic pressure as a function of one or more fluid outflow parameters of the surgical system, and calculate the intraocular pressure based on the estimated static pressure and the estimated dynamic pressure.

30. The system of claim 29, wherein the fluid outflow parameter is one or more selected from the group consisting of aspiration rate, vacuum rate, tip size, a compliance of tubing, a length of tubing, and an inside diameter of tubing.

31. The system of claim 27, further comprising a sensor system for determining at least one of the fluid inflow parameters.

32. The system of claim 29, further comprising a sensor system for determining at least one of the fluid outflow parameters.

33. The system of claim 27, wherein the surgical system comprises a handpiece combining irrigation, aspiration and emulsification capabilities.

34. The system of claim 27, wherein the processor is configured to control the surgical system so as to achieve a target intraocular pressure based on the calculated intraocular pressure.

35. The system of claim 34, wherein the processor is configured to control the surgical system so as to achieve the target intraocular pressure based on the calculated intraocular pressure by adjusting any one or more of irrigation flow rate, irrigation inflow pressure, vacuum rate and aspiration rate of the surgical system.

36. The system of claim 27, wherein the processor is configured to repeatedly calculate the intraocular pressure.

37. The system of claim 27, wherein the processor is configured to repeatedly calculate the intraocular pressure based at least on sensed aspiration rate of the surgical system.
